(19) United States
(12) Patent Application Publication
(10) Pub. No.: US 2012/0245663 A1
(43) Pub. Date: Sep. 27, 2012

(54) IMPLANTABLE MEDICAL DEVICE HAVING AN ADHESIVE SURFACE PORTION

(76) Inventors: Paul E. Zarembo, Vadnais Heights, MN (US); Shrojalkumar Desai, Vernon Hills, IL (US); Bruce A. Tockman, Scandia, MN (US); Christopher Hartemink, Shoreview, MN (US)

(21) Appl. No.: 13/428,957

(22) Filed: Mar. 23, 2012

Related U.S. Application Data
(60) Provisional application No. 61/467,255, filed on Mar. 24, 2011.

(51) Int. Cl. A61N 1/05

(52) U.S. Cl. 607/116

(57) ABSTRACT

An implantable medical device includes an adhesive surface portion provided over or formed on an outer surface thereof. The adhesive surface portion is capable of bonding to body tissue to secure and fixate the implantable medical device at a desired implantation location within the patient's body. The adhesive surface portion can be used in combination with other fixation mechanisms to secure and fixate the implantable medical device at the desired implantation location.

### CROSS-REFERENCE TO RELATED APPLICATIONS

This application claims the benefit under 35 U.S.C. section 119(e) to U.S. provisional application No. 61/467,255, entitled “Implantable Medical Device Having an Adhesive Surface Portion”, filed on Mar. 24, 2011, which is herein incorporated by reference in its entirety.

### TECHNICAL FIELD

The present disclosure relates to implantable medical devices and more particularly, to implantable medical devices having an adhesive surface portion for adhering the implantable medical device to body tissue.

### BACKGROUND

Leads, tubes, catheters, and stents that are implanted within a patient's body are typically held in place through the use of tines, sutures, overall shape of the device, or some other mechanical fixation feature. Sometimes these mechanical features alone are insufficient to prevent migration, disorientation, or dislodgement of the implanted device. Other methods or features may be necessary to enhance fixation of the implanted device.

### SUMMARY

Example 1 is a medical electrical lead that includes a lead body that extends from a proximal end to a distal end and that includes an outer surface when the lead body is implanted within a patient's body. At least one conductor extends through the lead body and is operatively connected to at least one electrode that is located within the lead body. An adhesive surface portion is positioned on the medical electrical lead and provides sufficient tissue adhesion to fixate the medical electrical lead to body tissue.

In Example 2, the medical electrical lead of Example 1 in which the adhesive surface portion includes sufficient tissue adhesive functional groups to fixate the medical electrical lead to body tissue.

In Example 3, the medical electrical lead of Example 2 in which the tissue adhesive functional groups include one or more of hydroxyl groups, carboxyl groups, amine groups and combinations thereof.

In Example 4, the medical electrical lead of Example 2 in which the adhesive surface portion includes at least one polymer selected from the group consisting of polysaccharides, polylactates, polyalkylene glycols, proteins, and combinations thereof.

In Example 5, the medical electrical lead of Example 2 in which the adhesive surface portion includes at least one polymer selected from the group consisting of poly(vinylamine), poly(ethyleneinime), poly(allyl-amine), polyethylene glycol, polyethylene glycol-co-aspartic acid), poly(lysine-co-lactide), poly(cysteine-co-lactide), polymethylmethacrylate, poly(2-aminoethylmethacylate) and combinations.

In Example 6, the medical electrical lead of any of Examples 2-5 in which the adhesive surface portion includes at least one polymer coating including the tissue adhesive functional groups.

In Example 7, the medical electrical lead of Example 6 in which the at least one polymer coating includes polymerizable and/or cross-linkable material adapted to undergo polymerization and/or cross-linking when contacted with body fluid or tissue.

In Example 8, the medical electrical lead of any of Examples 2-7 in which the adhesive surface portion is disposed on the outer surface of the lead body adjacent to the electrode.

In Example 9, the medical electrical lead of any of Examples 2-8 in which the adhesive surface portion is disposed on at least one passive or active fixation structure extending from the lead body.

In Example 10, the medical electrical lead of Example 1 in which the adhesive surface portion includes a naturally-derived tissue adhesive coating selected from the group consisting of chitosan, hyaluranon, collagen, collagen mimicking peptide, proteinous gelatin, alginate, glycosaminoglycan, arginine-glycine-aspartate, fibronectin, vitronectin, and extra-cellular matrices.

Example 11 is a medical electrical lead that includes a lead body extending from a proximal end to a distal end, the lead body including an outer surface adapted to be exposed to a body environment when implanted within a patient's body, the outer surface including a microtextured surface portion adapted to electrostatically bond to the body tissue with sufficient strength to fixate the outer surface of the lead body to body tissue. At least one conductor extends within the lead body from the proximal end in a direction toward the distal end and at least one electrode is located on the lead body and is operatively coupled to the at least one conductor.

In Example 12, the medical electrical lead of Example 11 in which the microtextured surface includes a plurality of adhesive hairs or fibers extending outwardly from the outer surface of the lead body that are adapted to electrostatically bond to the body tissue.

In Example 13, the medical electrical lead of Example 12 in which the adhesive hairs are formed of a material that is the same as that of the lead body.

In Example 14, the medical electrical lead of any of Examples 11-13, further including tissue adhesive functional groups disposed on the microtextured surface.

Example 15 is a method of implanting a medical electrical lead including a lead body extending from a proximal to a distal end, at least one conductor extending within the lead body, at least one electrode located on the lead body and operatively coupled to the at least one conductor, an adhesive surface portion, and a protective cover disposed over the adhesive surface portion. The medical electrical lead is implanted in a vessel lumen and the protective cover is removed to expose the adhesive surface portion. The medical electrical lead is manipulated so that the adhesive portion contacts and fixates to a vessel wall.

In Example 16, the method of Example 15 in which removing the protective cover includes dissolving the protective cover in bodily fluid.

In Example 17, the method of Example 15 in which removing the protective cover includes withdrawing the protective cover.

In Example 18, the method of Example 15 in which the protective cover includes a distal portion of the lead body, and removing the protective cover includes extending the adhesive surface portion out of the distal end of the lead.

In Example 19, the method of Example 15 in which the adhesive surface portion includes an expandable member.

In Example 20, the method of any of Examples 15-19 in which manipulating the medical electrical lead includes inflating a balloon or expanding a stent near the adhesive surface portion.

While multiple embodiments are disclosed, still other embodiments of the present disclosure will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a medical electrical lead according to an embodiment of the present disclosure.

FIG. 2A is a longitudinal, cross-sectional view and FIG. 2B is an end, cross-sectional view of a lead according to an embodiment of the present disclosure.

FIG. 3A is a schematic view of a portion of a lead in accordance with yet another embodiment of the present disclosure.

FIGS. 3B and 3C are close-up, schematic views of a portion of the lead shown in FIG. 3A according to an embodiment of the present disclosure.

FIGS. 4A and 4B are schematic views of a portion of a lead according to yet another embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Leads according to various embodiments of the present disclosure are suitable for sensing intrinsic electrical activity and/or applying therapeutic electrical stimuli to a patient. Exemplary applications include but are not limited to cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to some embodiments may be endocardial leads that are configured to be partially implanted within one or more chambers of the heart so as to sense electrical activity of the heart and to apply a therapeutic electrical stimulus to the cardiac tissue within the heart. Additionally, leads formed in accordance with the various embodiments of the present disclosure may be suitable for placement in a coronary vein adjacent to the left side of the heart so as to facilitate bi-ventricular pacing in a CRT or CRT-D system. Still additionally, leads formed according to embodiments of the present disclosure may be configured to be delivered intravascularly to deliver an electrical stimulation therapy to a nerve or other neurostimulation target.

While the embodiments described herein generally relate to leads, it will be understood by those of skill in the art that adhesive surface portions, according to the various embodiments of the present disclosure, can be applied to a variety of medical devices including, but not limited to the following: medical tubing, catheters, stents, vena cava filters, Bion sensors, suture sleeves and the like. Some sensors utilizing the adhesive surface portions discussed herein may be used for neurostimulation and thus may be deployed in or near the brain, nerve bundles or the spinal cord. In some embodiments, suture sleeves may be configured such that the suture sleeve may slide along a lead to a desired position. The interior of the suture sleeve may include an adhesive portion that bonds to the lead once crimped or squeezed in position. Similarly, the exterior of the suture sleeve may include one or more adhesive portions that are adapted to secure the suture sleeve to body tissue.

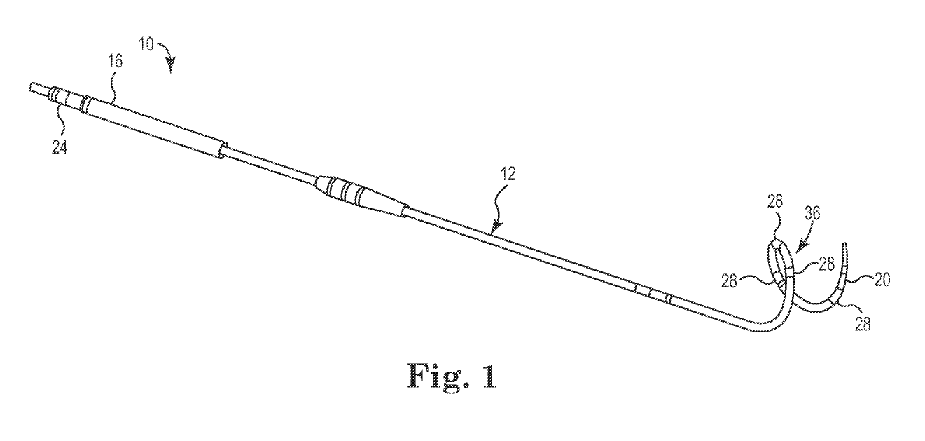

FIG. 1 is a perspective view of a medical electrical lead 10 according to an illustrative embodiment of the present disclosure. According to some embodiments, the lead 10 can be configured for implantation within a patient's heart or within a patient's neurovascular regions. The lead 10 includes an elongated, polymeric lead body 12 extending from a proximal end 16 to a distal end 20. In one embodiment, the distal end 20 has a tapered profile. The proximal end 16 of the lead body 12 is configured to be operatively connected to a pulse generator via a connector 24. At least one conductor (not shown) extends from the connector 24 through the lead body 12 to one or more electrodes 28 located at the distal end 20 of the lead 10. In some embodiments, the conductor may include coiled conductors, cable conductors or combinations thereof. In one embodiment, the lead 10 is a quadri-polar lead including one coiled conductor and three cable conductors. The coiled conductors can have either a co-radial or a co-axial configuration. In embodiments of the present disclosure employing multiple electrodes 28 and multiple conductors, each conductor is connected to an individual electrode 28 in a one-to-one manner allowing each electrode 28 to be individually addressable. In some embodiments, multiple electrodes 28 may be connected to a single conductor.

The lead body 12 is flexible, but substantially non-compressible along its length and, in some embodiments, has a circular cross-section. Other lead body cross-sections can be employed. According to one embodiment of the present disclosure, an outer diameter of the lead body 12 ranges from about 2 French to about 15 French. Additionally, the lead body 12 can be a multi-lumen lead body including at least two lumens. The lumens can have a variety of cross-sectional shapes and can be of the same or different sizes. The lumens facilitate passage of the conductor from the connector 24 to the electrode 28 and/or can receive a guiding element such as a guidewire or a stylet for delivery of the lead 10 to implant the lead 10 within a patient's heart. In some embodiments, the lead body 12 may include a single lumen.

The polymeric material used to form the lead body 12 can include a variety of different biocompatible polymeric materials, polymeric material blends, co-block polymers, co-polymers, and elastomers used to manufacture lead bodies known to those of skill in the art. Exemplary polymeric materials include, but are not limited to, silicone, polyurethane, polyethylene teraphthalate, polytetrafluoroethylene, and fluorinated ethylene propylene. Other exemplary materials suitable for use as lead body materials include, but are not limited to block co-polymer elastomers, polyurethane, polyurethane blends, polyurethane co-polymers, silicone rubbers, styrene-isobutylene-styrene (SIBS) co-polymers, and the like. In one embodiment, at least a portion of the lead body 12 is composed of silicone rubber.

The electrodes 28 can have any electrode configuration as is known in the art. According to one embodiment of the present disclosure, at least one electrode 28 can be a ring or partial ring electrode. According to another embodiment, at least one electrode 28 is a shocking coil. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

According to various embodiments, the lead body 12 can include one or more fixation members for securing and stabilizing the lead body 12 including the one or more electrodes 28 at a target site within a patient's body. The fixation member(s) can be active or passive. Examples of passive fixation include pre-formed portions of the lead body 12 such as, for example, a spiral 36, adapted to bear against the vessel walls and/or expandable tines provided at the distal end of the lead body 12. In some embodiments, the fixation member can be a screw-in fixation member. In other embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,444,334 which is herein incorporated by reference.

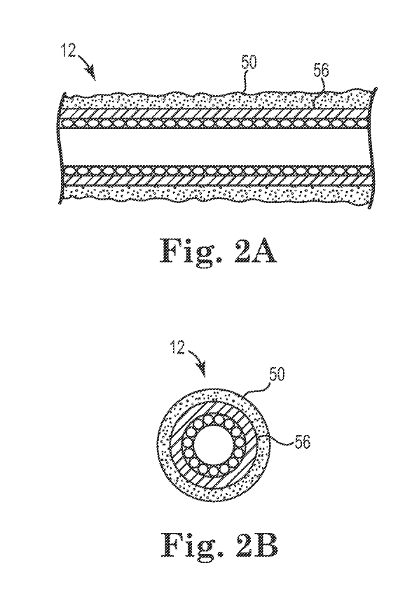

FIGS. 2A and 2B are cross-sectional views of a portion of a lead body 12, according to various embodiments of the present disclosure. As shown in FIGS. 2A and 2B, the lead body 12 includes an adhesive surface portion 50 formed on or provided over at least a portion of an outer surface 56 of the lead body 12. According to some embodiments of the present disclosure, the adhesive surface portion 50 is provided over the outer surface 56 of the lead body 12 such that the adhesive surface portion 50 extends from substantially the proximal end 16 to the distal end 20 of the lead body 12. According to other embodiments of the present disclosure, the adhesive surface portion 50 is provided at one or more discrete locations along the lead body 12. The adhesive surface portion 50 attaches, bonds, or otherwise secures the lead body 12 to the body tissue at one or more contact points to fixate the lead at a desired location within a patient's body.

In some embodiments, the adhesive surface portion 50 may be disposed adjacent or at least partially covering a conductive metal component such as one or more of the electrodes 28. In some embodiments, the adhesive surface portion 50 may only cover a portion of the electrode(s) 28 in order to permit electrical conduction. In some embodiments, the adhesive surface portion 50 may be formed to be thin enough or porous enough to permit electrical communication through the adhesive surface portion 50, particularly once in contact with bodily fluids such as blood.

In some embodiments, the adhesive surface portion 50 can be used in combination with other fixation members or mechanisms. For example, in one embodiment, the adhesive surface portion 50 can be provided over the outer surface 56 of a pre-formed portion of the lead body 12 such as, for example, a spiral (FIG. 1) adapted to contact and bear against the vessel walls to secure and stabilize the lead 10 at a desired location within the patient's body. Additionally, the adhesive surface portion 50 can be provided over the outer surface of any expandable tines, expandable loops, or an expandable stent-like member used to fixate the lead 10 at the desired location within the body. In one embodiment, the expandable stent-like member is provided over the lead body 12 or is adapted to be deployed from distal end of the lead body 12. In another embodiment, the stent-like member can be delivered separately from and along side the lead body 12 to secure and stabilize the lead at the desired location. In still other embodiments, the adhesive surface portion 50 can be provided over the outer surface of a fixation collar or an expandable portion of the lead body 12. In yet another embodiment, the adhesive surface portion 50 can be applied to a mesh structure provided over the lead body 12 and configured to contact body tissue to facilitate fibrous tissue growth. In this embodiment, the adhesive surface portion 50, applied to an outer surface of the mesh, fixates the lead 10 at a desired location until fibrous tissue ingrowth into the mesh occurs, chronically fixating the lead 10 at that location in the patient's body. The adhesive surface portion 50 may include a relatively permanent adhesive material or treatment, or a temporary/biodegradable treatment.

According to one embodiment, the adhesive surface portion 50 includes tissue-adhesive functional groups formed on the outer surface 56 of the lead body 12 at one or more locations, as described herein. Suitable tissue-adhesive functional groups include any functional group that is capable of reacting with the amine groups, thiol groups, and/or other nucleophilic groups present in the proteinaceous tissue surface so as to form covalent bonds between the tissue-adhesive functional groups on the outer surface 56 of the lead body 12 and the body tissue at the implantation site. Exemplary tissue-adhesive functional groups include but are not limited to hydroxyl (—OH) groups, amine (—NH₂) groups, carboxyl (—COOH) groups, cyanoacrylate groups, and functionalized cyanoacrylate groups. In one embodiment, the tissue-adhesive functional groups are carboxyl (—COOH) groups.

The tissue-adhesive functional groups can be formed on the outer surface 56 using a variety of surface treatment methods. The tissue-adhesive functional groups are formed such that they extend away from the outer surface 56 of the lead body 12. Useful surface treatment methods include chemical reaction, plasma polymerization, spray coating, dip coating, solvent dipping, and absorption. In one embodiment, the outer surface 56 of the lead body is functionalized to include one or more tissue-adhesive functional groups by chemical plasma vapor deposition. In another embodiment, the tissue-adhesive function groups can be formed via chemical reaction with the outer surface 56 of the lead body 12.

According to one embodiment, the adhesive surface portion 50 includes a thin layer of a tissue-adhesive coating or sleeve applied to the outer surface 56 of the lead body 12 at one or more locations, as described above. For example, the tissue-adhesive coating may include tissue-adhesive material(s) that readily bond to the tissue surface that is rich in protein groups containing amine groups, thiol groups, and other nucleophilic groups.

According to certain embodiments, the tissue-adhesive coating includes a tissue-adhesive material dispersed within a polymerizable and/or cross-linkable material. The tissue-adhesive material includes any functional group that is capable of reacting with the amine groups and/or thiol groups present in the tissue surface so as to form covalent bonds between the tissue-adhesive coating and the tissue. Exemplary tissue-reactive groups include but are not limited to hydroxyl groups, amine groups, and carboxyl groups. Additional exemplary tissue-reactive functional groups include imido ester, p-nitrophenyl carbonate, N-hydroxysuccinimide (NHS) ester, epoxide, isocyanate, acrylate, vinyl sulfone, orthopyridyl-disulfide, maleimide, aldehyde, iodoacetamide, and others. In one embodiment, the tissue-reactive group is N-hydroxysuccinimide (NHS) ester. Exemplary polymerizable and/or cross-linkable materials include permanent and biodegradable polysaccharides, polylactates, polyalkylene glycols, proteins such as, for example, albumin, and derivatives thereof. Additional exemplary polymerizable and/or cross-linkable materials include poly(vinylamine), poly(ethyleneinime), poly(allyl-amine), polyethylene glycol, polyethylene glycol-co-aspartic acid), poly(lysine-co-lactide), poly(cysteine-co-lactide), polymethylmethacrylate, and poly(2-aminoethylmethacylate).

In one embodiment, the tissue adhesive coating can be formed by first combining the tissue adhesive material(s) with a polymerizable and/or cross-linkable material and then applying it to an outer surface 56 of the lead body 12. The tissue adhesive coating can be applied to the outer surface 56 of the lead body 12 using a variety of techniques including brush coating, dip coating, and the like. In another embodiment, the tissue adhesive material can be combined with the polymerizable and/or cross-linking material and polymerized and/or cross-linked with the outer surface 56 of the lead body 12. In some embodiments, the polymerizable and/or cross-linkable material forming a part of the tissue adhesive coating undergoes polymerization and/or cross-linking upon hydration when contacted with body fluid upon implantation. Additional tissue-adhesive coating materials are shown and described in US Publication No. 2009/0044895, entitled “Tissue Adhesive Materials,” and U.S. Pat. No. 7,727,547, entitled “Tissue Adhesive Formulations,” both of which are incorporated herein by reference in theft entireties for all purposes.

The amount of tissue-adhesive material in the coating can range from about 5 wt. % to about 50 wt. % (weight of the tissue adhesive material/total weight of the coating). In other embodiments, the amount of tissue adhesive material is at least 50 wt. %. In still yet other embodiments, the amount of tissue-adhesive material in the coating can range from about 55 to about 75 wt. %; 45 to about 65 wt. %; about 35 to about 55 wt. %; about 25 to about 45 wt. %; about 15 to about 35 wt. %; about 5 to about 25 wt. %; and from about 1 to about 15 wt. %.

According to another embodiment, the tissue-adhesive coating can include one or more synthetic or naturally-derived materials known to exhibit tissue adhesive properties. Exemplary naturally-derived materials exhibiting tissue adhesive properties include but are not limited to, the following: chitosan, hyaluranon, collagen, MATRIGEL™ (proteinous gelatin), gelatin, alginate, and glycosaminoglycan. Additional materials having tissue adhesive properties include pro-healing peptides such as arginie-glycine-aspartate (RGD) and GFOGER (collagen mimicking peptide), fibronectin and vitronectin, and extra-cellular matrix molecule collagen. Hydroxyapatite can also be used to form a tissue-adhesive coating. In some embodiments, these compounds can be reacted with the outer surface 56 of the lead body 12 to provide an adhesive surface portion 50 at one or more locations along the lead body 12. In another embodiment, these compounds can be admixed with a carrier material and coated onto the outer surface 56 of the lead body 12. Suitable carrier materials include synthetic and naturally-derived polymers that do not interfere with the adhesive properties of the naturally-derived materials. The amount of naturally-derived adhesive material in the coating can range from about 1 to about 100 wt. %. In other embodiments, the amount of naturally-derived adhesive material in the coating may range from about 90 to about 100 wt. %; about 80 to about 100 wt. %; about 75 to about 90 wt. %; about 65 to about 85 wt. %; about 55 to about 75 wt. %; 45 to about 65 wt. %; about 35 to about 55 wt. %; about 25 to about 45 wt. %; about 15 to about 35 wt. %; about 5 to about 25 wt %; and from about 1 to about 15 wt. %.

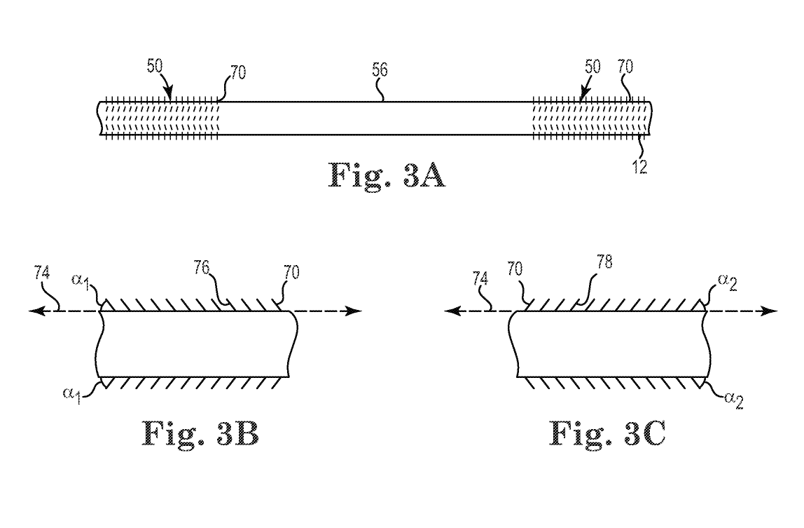

According to still other embodiments, the adhesive surface portion 50 can include a microtexture formed, for example from a plurality of microscopic adhesive fibers or hairs 70 provided over and/or formed on an outer surface 56 of the lead body 12 as shown schematically in FIGS. 3A-3C. The adhesive hairs 70 act in a similar manner to the setae on the bottom of a gecko's feet. Like the setae found on the bottom of a gecko's feet, the material used to form the adhesive hairs 70 is not necessarily itself adhesive. Rather, and in some embodiments, each individual hair 70 may have its own electrostatic charge. When brought into contact with a surface of an object such as for example, the inner surface of a body vessel in which the lead is implanted, a charge difference may be created between each individual hair and the vessel surface, resulting in an attractive force. This attractive force is referred to as the Van der Waal force, and provides the adhesive hairs 70 with their collective adhesive properties. Various materials, often referred to as “Gecko Tape,” have been successfully tested in various medical applications.

As shown in FIGS. 3A-3C, the adhesive hairs 70 extend away from the outer surface 56 of the lead body 12. In one embodiment, the adhesive hairs 70 terminate in a plurality of finer frond-like elements (not shown), which can facilitate enhanced contact area between the individual hairs 70 and the body tissue. In some embodiments, each individual adhesive hair 70 can have a height ranging from about 0.5 microns to about 8 mm and a diameter ranging from about 2 microns to about 1000 microns. In some embodiments, the adhesive hairs 70 may be spaced apart from one another on the outer surface 56 of the lead body 12, and may have a packing density of at least 500 hafts per square centimeter. Together, the adhesive hairs 70 may provide sufficient adhesion to secure and stabilize the lead body 12 at a desired implantable location. However, together the adhesive hairs 70 are not so adhesive that the lead body 12 cannot be repositioned and/or removed from the patient's body. In some embodiments, the adhesive hairs 70 can be used to initially adhere the lead to the body tissue until another adhesive surface portion, such as described above, can be activated. According to additional embodiments, the adhesive surface portion 50 including the adhesive hairs 70 are further coated with a biocompatible coating such as a biocompatible polymer material.

The adhesive hairs 70 can be hydrophilic or hydrophobic, and can be fabricated from a variety of materials. Exemplary materials include polyesters, polyamides, polyurethanes, polysiloxanes, polydimethylsiloxane, silicone rubbers, and the like. In one embodiment, the adhesive hairs 70 are formed from the same material as is the lead body 12. In certain embodiments, the adhesive hairs 70 are hollow. The adhesive hafts can be made by a variety of methods including lithography, nanomolding using a template, etching (including on etching and plasma etching) self-assembling mono-layers (SAMs), atomic force microscopy, and the like.

According to various embodiments, the adhesive hairs 70 are adapted to adhere to a surface when oriented in a first direction and are adapted to release from a surface when oriented in a second direction. For example, when oriented in a first direction 76 at a first angle α₁ relative to the plane 74 of the outer surface 56 of lead body 12 (FIG. 3B), the adhesive hairs 70 are capable of adhering to the inner wall of the vessel in which the lead is deployed to secure and fixate the lead at the desired location within the vessel. When oriented in a second direction 78 at a second angle α₂ relative to the plane 74 of the outer surface 56 of the lead body 12 different from the first direction (FIG. 3C), the adhesive hairs 70 release from the vessel surface. In some embodiments, the lead may be repositioned or removed by applying a force sufficient to overcome the attractive forces between the adhesive hairs 70 and the surface to which they are attracted. This feature facilitates repositioning and/or removal of the lead after implantation, before tissue ingrowth around the lead has occurred.

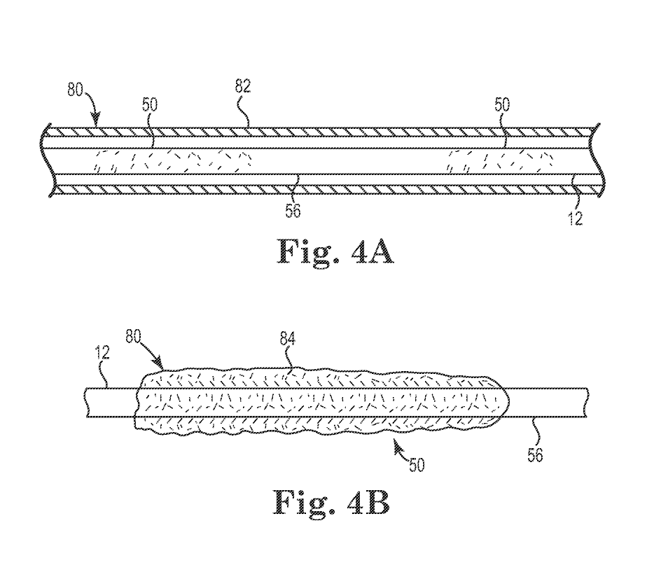

In some embodiments, leads including the adhesive surface portions 50, as describe herein according to the various embodiments, may include a protective covering that prevents adherence to body tissue prior to implantation and/or during implantation while the lead is still being positioned. As shown for example in FIGS. 4A and 4B, the lead body 12 may include a protective covering 80 that is schematically shown as covering the adhesive surface portion 50.

In one embodiment, as shown in FIG. 4A, the protective covering 80 is a removable outer sheath 82 that can be withdrawn or otherwise removed once the lead body 12 has been positioned as desired. In another embodiment, as shown in FIG. 4B, the protective covering 80 may be a dissolvable coating 84 such as, for example, a mannitol or PEG (polyethylene glycol) coating. According to various embodiments, the protective covering 80 is capable of retaining any pre-formed portion of the lead body 12 or expandable fixation feature in a collapsed configuration during delivery of the lead 10. Upon removal of the protective covering 80, contact between the adhesive surface portion on the lead body 12 and the tissue at the implantation site occurs. Additionally, removal of the protective covering 80 results in expansion of any pre-formed portion of the lead body 12 or expandable fixation features.

Leads including the adhesive surface portions 50, described herein, can be delivered to the desired implantation location using a variety of well known techniques. The leads can be delivered using a guide catheter, a guidewire, and/or a stylet typically employed for this purpose. Once delivered to the desired location, the protective covering is withdrawn or dissolved and the lead 10 is manipulated so that the tissue adhesive portion 50 is placed into contact with the vessel wall. For example an expandable balloon or stent may be used to move the tissue adhesive portion 50 into the wall. If the tissue adhesive portion 50 is disposed, for example, on an expandable tine to similar structure, the tissue adhesive portion may come into contact with the vessel wall as soon as the protective covering 80 is removed, or the expandable structure is otherwise expanded relative to the lead 10.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical electrical lead comprising:
a lead body extending from a proximal end to a distal end, the lead body comprising an outer surface when implanted within a patient's body;
at least one conductor extending within the lead body;
at least one electrode located within the lead body and operatively coupled to the at least one conductor; and
an adhesive surface portion positioned on the medical electrical lead and providing sufficient tissue adhesion to fixate the medical electrical lead to body tissue.

2. The medical electrical lead of claim 1, wherein the adhesive surface portion comprises sufficient tissue adhesive functional groups to fixate the medical electrical lead to body tissue.

3. The medical electrical lead of claim 2, wherein the tissue-adhesive functional groups are selected from the group consisting of hydroxyl groups, carboxyl groups, amine groups and combinations.

4. The medical electrical lead of claim 2, wherein the adhesive surface portion comprises at least one polymer selected from the group consisting of polysaccharides, polylactates, polyalkylene glycols, proteins, and combinations.

5. The medical electrical lead of 2, wherein the adhesive surface portion comprises at least one polymer selected from the group consisting of poly(vinylamine), poly(ethyleneinime), poly(allyl-amine), polyethylene glycol, polyethylene glycol-co-aspartic acid), poly(lysine-co-lactide), poly(cysteine-co-lactide), polymethylmethacrylate, poly(2-aminoethylmethacylate) and combinations.

6. The medical electrical lead of claim 2, wherein the adhesive surface portion comprises at least one polymer coating including the tissue adhesive functional groups.

7. The medical electrical lead of claim 6, wherein the coating comprises polymerizable and/or cross-linkable material adapted to undergo polymerization and/or cross-linking when contacted with body fluid or tissue.

8. The medical electrical lead of claim 1, wherein the adhesive surface portion is disposed on the outer surface of the lead body adjacent to the electrode.

9. The medical electrical lead of claim 1, wherein the adhesive surface portion is disposed on at least one passive or active fixation structure extending from the lead body.

10. The medical electrical lead of claim 1, wherein the adhesive surface portion comprises an adhesive surface portion including a naturally-derived tissue adhesive coating selected from the group consisting of chitosan, hyaluranon, collagen, collagen mimicking peptide, proteinous gelatin, alginate, glycosaminoglycan, arginine-glycine-aspartate, fibronectin, vitronectin, and extra-cellular matrices.

11. A medical electrical lead comprising:
a lead body extending from a proximal end to a distal end, the lead body including an outer surface adapted to be exposed to a body environment when implanted within a patient's body, the outer surface including a microtextured surface portion adapted to electrostatically bond to the body tissue with sufficient strength to fixate the outer surface of the lead body to body tissue;
at least one conductor extending within the lead body from the proximal end in a direction toward the distal end; and
at least one electrode located on the lead body and operatively coupled to the at least one conductor.

12. The medical electrical lead of claim 11, wherein the microtextured surface comprises a plurality of adhesive hairs or fibers extending outwardly from the outer surface of the lead body adapted to electrostatically bond to the body tissue.

13. The medical electrical lead according to claim 11, wherein the adhesive hairs are formed of a material that is the same as that of the lead body.

14. The medical electrical lead according to claim 11, further comprising tissue adhesive functional groups disposed on the microtextured surface.

15. A method of implanting a medical electrical lead comprising a lead body extending from a proximal to a distal end, at least one conductor extending within the lead body, at least one electrode located on the lead body and operatively coupled to the at least one conductor, an adhesive surface portion, and a protective cover disposed over the adhesive surface portion, the method comprising:
implanting the medical electrical lead in a vessel lumen;
removing the protective cover to expose the adhesive surface portion; and
manipulating the medical electrical lead so that the adhesive surface portion contacts and fixates to a vessel wall.

16. The method according to claim 15, wherein removing the protective cover comprises dissolving the protective cover in bodily fluid.

17. The method according to claim 15, wherein removing the protective cover comprises withdrawing the protective cover.

18. The method according to claim 15, wherein the protective cover comprises a distal portion of the lead body, and removing the protective cover comprises extending the adhesive surface portion out of the distal end of the lead.

19. The method according to claim 15, wherein the adhesive surface portion comprises an expandable member.

20. The method according to claim 15, wherein the manipulating step comprises inflating a balloon or expanding a stent near the adhesive surface portion.
